(19) United States
(12) Patent Application Publication
(10) Pub. No.: US 2026/0364326 A1
(43) Pub. Date: Nov. 19, 2026

(54) DRUG DELIVERY DEVICE

(71) Applicant: Kindeva Drug Delivery L.P., Woodbury, MN (US)

(72) Inventor: Richard Brewer, Loughborough (GB)

(21) Appl. No.: 19/138,515

(22) PCT Filed: Dec. 4, 2023
(86) PCT No.: PCT/US2023/082372

§ 371 Date: Jun. 12, 2025

Related U.S. Application Data
(60) Provisional application No. 63/433,257, filed on Dec. 16, 2022.

(51) Int. Cl. A61M 37/00

(52) U.S. Cl. CPC A61M 37/0015; A61M 2037/0023; A61M 2037/0038; A61M 2037/0046; A61M 2037/0053; A61M 2037/0061

(57) ABSTRACT

Various embodiments of a drug delivery device (10, 200, 300) are disclosed. The device includes a backing layer (12, 312) having a first major surface (14, 314) and a second major surface (15) and a plurality of discrete arrays (16, 216, 316) disposed on the first major surface of the backing layer. The plurality of discrete arrays includes a first discrete array (18, 318) that has a first substrate (20, 220, 320) and first microneedles (22, 322) disposed on the first substrate, and a second discrete array (24, 216, 324) that has a second substrate (26, 220, 326) and second microneedles (28, 328) disposed on the second substrate.

This application claims the benefit of U.S. Provisional Application No. 63/433,257, filed Dec. 16, 2022, the disclosure of which is incorporated by reference herein in its entirety.

### BACKGROUND

Transdermal and topical drug delivery can be used for therapeutic treatment, but the number of molecules that can be effectively delivered using these routes can be limited by the barrier properties of skin. The main barrier to transport of molecules through the skin is the stratum corneum (the outermost layer of the skin).

Various skin treatment methods have been proposed to increase the permeability or porosity of the outermost skin layers, such as the stratum corneum, thus enhancing drug delivery through or into those layers. The stratum corneum is a complex structure of compact keratinized cell remnants separated by lipid domains. The stratum corneum is formed of keratinocytes, which includes the majority of epidermal cells, which lose their nuclei and become corneocytes. These dead cells make up the stratum corneum, which has a thickness of only about 10-30 microns and protects the body from invasion by exogenous substances and the outward migration of endogenous fluids and dissolved molecules. Various skin treatment methods include the use of microneedles, laser ablation, RF ablation, heat ablation, sonophoresis, iontophoresis, or combinations of these treatment methods.

Devices including arrays of relatively small structures, sometimes referred to as microneedles or micro-pins, have been disclosed for use with the delivery of therapeutic agents and other substances through the skin and other surfaces. The devices can be pressed against the skin to pierce the stratum corneum such that the therapeutic agents and other substances can sequentially or simultaneously pass through that layer and into the tissues below. Microneedles of these devices pierce the stratum corneum upon contact, making a plurality of microscopic slits that serve as passageways through which molecules of active components can be delivered into the body. In delivering an active component, the microneedle device can be provided with a reservoir for temporarily retaining an active component in liquid form prior to delivering the active component through the stratum corneum. In some constructions, the microneedles can be hollow to provide a liquid flow path directly from the reservoir and through the microneedles to enable delivery of the therapeutic substance through the skin. In alternate constructions, active component(s) may be coated on the microneedle array and delivered directly through the skin after the stratum corneum has been punctured. Further, one or more constructions of microneedle arrays can include dissolvable microneedles.

Microneedle arrays and patches can be deployed with an applicator. The microneedle arrays and patches are generally used once and then discarded. The applicator devices can be repeatedly reloaded with new microneedle arrays and patches.

### SUMMARY

In general, the present disclosure provides various embodiments of a drug delivery device. The device can include a backing layer and a plurality of discrete microarrays disposed on the backing layer. The plurality of discrete microarrays can include at least first and second discrete arrays. Each of the first and second discrete arrays includes microneedles disposed on a substrate. In one or more embodiments, the first discrete array includes a first characteristic and the second discrete array includes a second characteristic different from the first characteristic.

In one aspect, the present disclosure provides a drug delivery device that includes a backing layer having a first major surface and a second major surface, and a plurality of discrete arrays disposed on the first major surface of the backing layer. The plurality of discrete arrays includes a first discrete array that has a first substrate and first microneedles disposed on the first substrate, and a second discrete array that has a second substrate and second microneedles disposed on the second substrate.

In another aspect, the present disclosure provides a method of forming a drug delivery device. The method includes forming a plurality of discrete arrays, where the plurality of discrete arrays includes a first discrete array having a first substrate and first microneedles disposed on the first substrate, and a second discrete array having a second substrate and second microneedles disposed on the second substrate. The first discrete array includes a first characteristic and the second discrete array includes a second characteristic different from the first characteristic. The method further includes disposing the plurality of discrete arrays on skin of a user.

All headings provided herein are for the convenience of the reader and should not be used to limit the meaning of any text that follows the heading, unless so specified.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

In this application, terms such as “a,” “an,” and “the” are not intended to refer to only a singular entity but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

These and other aspects of the present disclosure will be apparent from the detailed description below. In no event, however, should the above summaries be construed as limitations on the claimed subject matter, which subject matter is defined solely by the attached claims, as may be amended during prosecution.

### BRIEF DESCRIPTION OF THE DRAWINGS

Throughout the specification, reference is made to the appended drawings, where like reference numerals designate like elements, and wherein:

FIG. 1 is a schematic plan view of one embodiment of a drug delivery device.

FIG. 2 is a schematic cross-section view of the drug delivery device of FIG. 1.

FIG. 3 is a schematic top perspective view of a discrete array of the drug delivery device of FIG. 1.

FIG. 4 is a schematic bottom plan view of the discrete array of FIG. 3.

FIG. 5 is a schematic cross-section view of a portion of the discrete array of FIG. 3.

FIG. 6 is a schematic bottom perspective view of another embodiment of a drug delivery device.

FIG. 7 is a schematic top plan view of another embodiment of a drug delivery device.

FIG. 8 is a schematic side view of one embodiment of a microarray applicator that can be utilized to apply the drug delivery device of FIG. I to skin of a user.

FIG. 9 is a schematic partial cross-section view of the microarray applicator of FIG. 8.

FIG. 10 is a flowchart of one method of forming the drug delivery device of FIG. 1.

### DETAILED DESCRIPTION

In general, the present disclosure provides various embodiments of a drug delivery device. The device can include a backing layer and a plurality of discrete microarrays disposed on the backing layer. The plurality of discrete microarrays can include at least first and second discrete arrays. Each of the first and second discrete arrays includes microneedles disposed on a substrate. In one or more embodiments, the first discrete array includes a first characteristic and the second discrete array includes a second characteristic different from the first characteristic.

Typical microneedle arrays include a monolithic substrate and a plurality of microneedles disposed on the substrate. The microneedles tend to be uniform in size, shape, and dimension. One or more embodiments of drug delivery devices described herein can provide various advantages over currently available devices. For example, one or more embodiments of drug delivery devices can include a plurality of discrete arrays each including a substrate and microneedles disposed on the substrate. As used herein, the term “discrete” means that each array is manufactured separately as an individual component or element. These discrete arrays can then be combined either directly on skin of a user or on a backing layer and then disposed on the skin to provide the drug delivery device. Because the device includes discrete arrays, the overall size of the drug delivery device can be configurable to provide differing overall shapes and dimensions. Further, one or more of the discrete arrays can exhibit one or more characteristics that are different from one or more characteristics of one or more additional discrete arrays of the device. For example, one or more discrete arrays of the device can each include a first number of microneedles, and one or more additional discrete arrays can each include a second number of microneedles that is different from the first number of microneedles. These discrete arrays can be easier to control during manufacture (e.g., by molding or casting) because of their smaller dimensions when compared to standard unitary arrays. Further, the device can be more conformable to skin than traditional monolithic devices as the discrete arrays allow the device to form to contours of the application location, which may allow for more uniform needle penetration than would be achieved with a single rigid array.

In discussing the drug delivery devices and applicators of the present disclosure, the term “downward,” and variations thereof, is sometimes used to describe the direction in which microneedles are pressed into skin, and “upward” to describe the opposite direction. However, those of skill in the art will understand that the drug delivery device and applicator can be used where the microneedles are pressed into skin at an angle to the direction of the earth's gravity, or even in a direction contrary to that of the earth's gravity, and these terms are only used for simplicity and clarity to describe relative directions.

The term “transdermally,” and variations thereof, is generally used to refer to any type of delivery of an active ingredient that crosses any portion of skin. That is, transdermally can generally include systemic delivery (i.e., where the active ingredient is transported across, or substantially through, the dermis such that the active ingredient is delivered into the bloodstream), as well as intradermal delivery (i.e., where the active ingredient is transported partially through the dermis, e.g., across the outer layer (stratum corneum) of the skin, where the active ingredient is delivered into the skin, e.g., for treating psoriasis or for local anesthetic delivery). That is, transdermal delivery as used herein includes delivery of an active or placebo ingredient that is transported across at least a portion of skin (but not necessarily all of the layers of skin), rather than merely being topically applied to an outer layer of the skin.

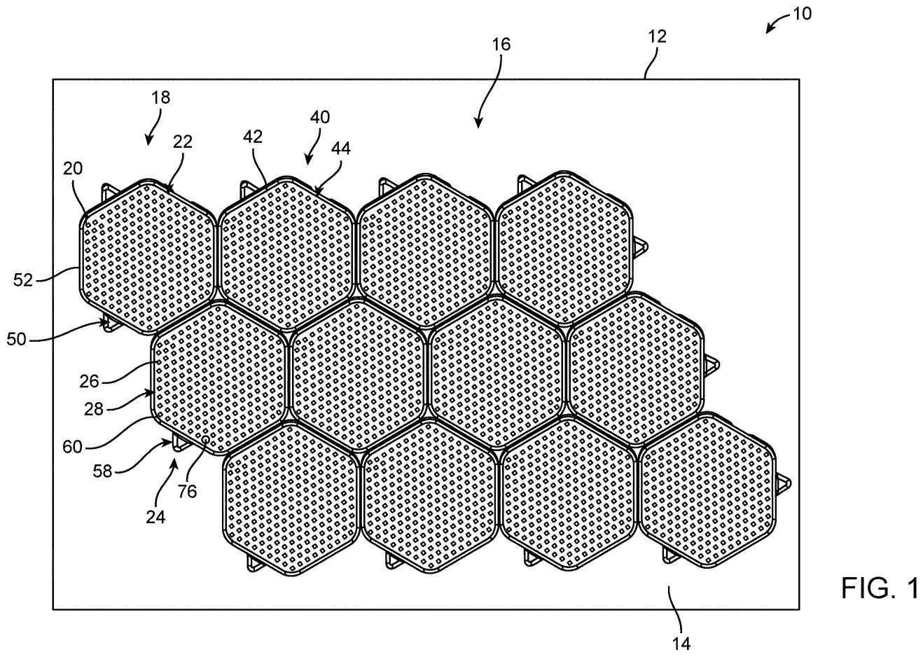

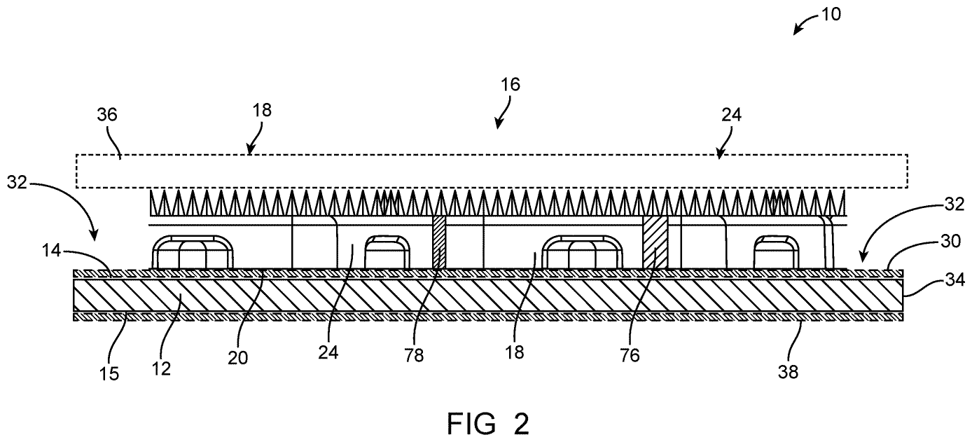

FIGS. 1-2 are plan and side cross-section views of one embodiment of a drug delivery device 10. The device 10 includes a backing layer 12 that includes a first major surface 14 and a second major surface 15, and a plurality of discrete arrays 16 disposed on the first major surface of the backing layer. The plurality of discrete arrays 16 includes a first discrete array 18 that includes a first substrate 20 and first microneedles 22 disposed on the first substrate, and a second discrete array 24 that includes a second substrate 26 and second microneedles 28 disposed on the second substrate.

The device 10 can be utilized for any suitable application to provide one or more active pharmaceutical ingredients to the user. In one or more embodiments, the device 10 can include a transdermal patch as described, e.g., in U.S. Pat. No. 9,375,510 to DiZio et al. and entitled TRANSDERMAL ADHESIVE COMPOSITIONS, DEVICES AND METHODS. Further, in one or more embodiments, the device 10 can include a topical patch, e.g., patch that is disposed on a surface of the user's skin and provides one or more active pharmaceutical ingredients topically to the skin as described in U.S. Pat. No. 6,096,334 to Rolf et al. and entitled ADHESIVE PATCH FOR APPLYING MEDICATION TO THE SKIN AND METHOD.

Further, the drug delivery device 10 can be disposed individually or in roll form in any suitable pouch for storage, e.g., a foil lined pouch. In one or more embodiments, the drug delivery device 10 can be provided in a rolled or stacked form suitable for use with a dispensing apparatus.

The drug delivery device 10 can include any suitable device or devices that can be disposed on the skin of the patient. Although depicted as a single device 10, the drug delivery device can include two or more individual delivery devices that can be disposed on the backing layer 12 or on separate backing layers. Further, the device 10 can be disposed on the backing layer 12 using any suitable technique. For example, as shown in FIG. 2, an adhesive layer 30 can be disposed on the first major surface 14 of the backing layer 12 such that the adhesive layer is between the plurality of discrete arrays 16 and the first major surface of the backing layer. The adhesive layer 30 can include any suitable adhesive. In one or more embodiments, the adhesive layer 30 can include any polymer, or combination of polymers, which provides a desirable combination of adhesion to the skin and delivery of the one or more active pharmaceutical ingredients. Exemplary suitable polymers include, but are not limited to, acrylates, silicones, polyisobutylenes, and mixtures thereof. In one or more embodiments, the adhesive layer 30 can include a silicone adhesive composition. Other embodiments of suitable adhesive compositions are described, e.g., in U.S. patent application Ser. No. 63/048,516, (Attorney Docket No. 83037US002), entitled DRUG DELIVERY DEVICE FOR DELIVERY OF CLOBETASOL PROPIONATE. Although depicted as included a single adhesive layer 30, the device 10 can include any suitable number of adhesive layers disposed between the discrete arrays 16 and the backing layer 12.

In one or more embodiments, one or more portions 32 of the adhesive layer 30 disposed adjacent to a periphery 34 of the backing layer 12 can be exposed (i.e., the portions 32 do not include a discrete array disposed thereon) such that the adhesive layer is configured to be adhered to the skin of the user utilizing these one or more portions. In one or more embodiments, the one or more portions 32 can be disposed between the discrete arrays 16 and the periphery 34 of the backing layer 12.

The backing layer 12 can include any suitable material, e.g., at least one of an inorganic (e.g. metallic) or organic (i.e., polymeric) material. Suitable materials include conventional flexible backing materials used for pressure sensitive adhesive tapes, including, for example, polyethylene (particularly low density polyethylene, linear low density polyethylene, metallocene polyethylenes, or high density polyethylene), polypropylene, polyesters such as polyethylene terephthalate, randomly oriented nylon fibers, ethylene-vinyl acetate copolymer, polyurethane, natural fibers such as rayon and the like. Suitable materials also include materials that are layered, such as polyethylene terephthalate-aluminum-polyethylene composites. In one or more embodiments, the backing layer 12 may be formed from low density polyethylene (LDPE) film, which is a low modulus film relative to other transdermal backings. One exemplary low density polyethylene film is a 1.7 mil LDPE film (COTRAN, 3M Corp., St. Paul, MN). Further, the backing layer 12 can be treated using any suitable technique or techniques, e.g., the techniques described in co-filed U.S. patent application Ser. No. 63/048,516 (Attorney Docket No. 83037US002), entitled DRUG DELIVERY DEVICE FOR DELIVERY OF CLOBETASOL PROPIONATE. In one or more embodiments, the backing layer 12 can include one or more stiffening layers such as a polycarbonate layer that provides rigidity to the device 10.

The backing layer 12 can further include indicia or printed images disposed on or in the layer. For example, a decorative design can be disposed on at least one of the first or second major surfaces 14, 15 or within the backing layer 12. Further, a separate layer that includes indicia or printed images can be disposed on the backing layer 12 using any suitable technique.

Further, the backing layer 12 can take any suitable shape in a plane parallel to its first major surface 14. In one or more embodiments, the backing layer 12 can include a polygonal shape in the plane parallel to the first major surface 14 of the backing layer. In one or more embodiments, one or more perforations can be disposed in the backing layer 12 such that one or more portions of the backing layer can be removed to provide any suitable shape of patch segment. Exemplary patch segments are further described in one or more embodiments of PCT Patent Publication No. WO 2022/010837, entitled DRUG DELIVERY DEVICE INCLUDING PATCH SEGMENTS. When removing one more portions the backing layer 12 along such perforations, one or more discrete arrays 16 can also be removed to tailor the shape or configuration of the arrays.

In one or more embodiments, the drug delivery device 10 can also include an optional release liner 36 that can be disposed over the discrete arrays 16 such that these discrete arrays are disposed between the release liner and the backing layer 12. The release liner 36 can include any suitable material and take any suitable shape. Further, the release liner 36 can be connected to at least one of the backing layer 12 or microneedle arrays 16 using any suitable technique. Although depicted as including a single layer, the release liner 36 can include any suitable number of layers that include any suitable materials. In one or more embodiments, an additional release liner 38 can be disposed over the second major surface 15 of the backing layer as shown in FIG. 2. The optional second release liner 38 can include any suitable material have any suitable number of layers. The release liner 36 can be removed from the device 10 prior to application of the device onto the skin of the user. Further, the optional second release line 38 can be removed either prior to or following application of the device 10 to the skin.

Disposed on the first major surface 14 of the backing layer 12 is the plurality of discrete arrays 16. In one or more embodiments, the discrete arrays 16 can be disposed directly on and in contact with the first major surface 14 of the backing layer 12. In one or more embodiments, the discrete arrays 16 can be disposed on the adhesive layer 30 and connected to the backing layer with the adhesive layer.

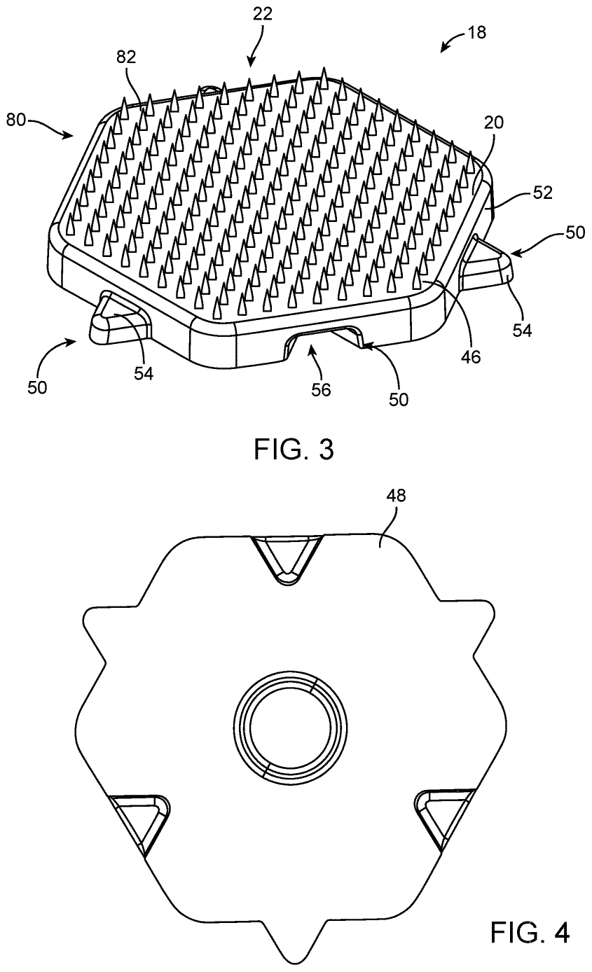

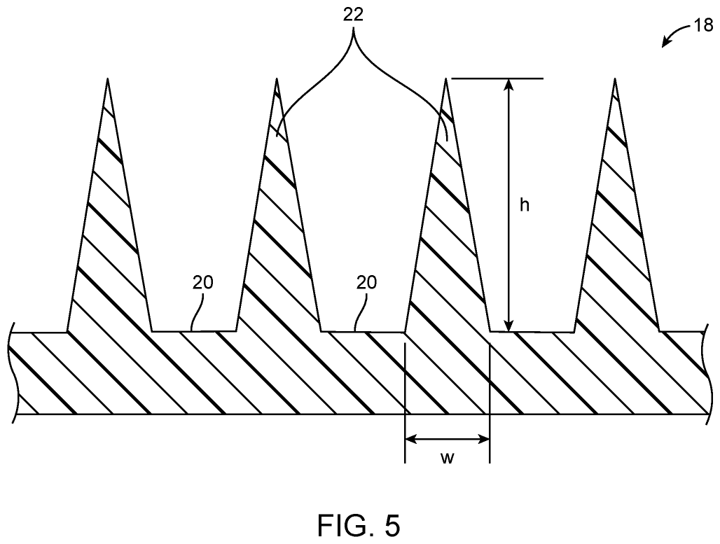

The discrete arrays 16 can include any suitable type of microneedle array. In one or more embodiments, each discrete array 16 can include a substrate and microneedles disposed on the substrate. For example, FIGS. 3-4 are various view of the first discrete array 18 that includes the first substrate 20 and first microneedles 22 disposed on the first substrate. The first substrate 20 includes a first major surface 46 and a second major surface 48. The first microneedles 22 can be disposed on the top surface 46 of the first substrate. The first microneedles 22 can be disposed in any suitable arrangement. Further, one or more portions of the of the first substrate 20 can be free of microneedles. For example, as shown in FIG. 3, a first portion 80 of the first substrate 20 can include an arrangement of first microneedles 22 and a second portion 82 of the first substrate does not include first microneedles. Each of the first and second portions 80, 82 can have any suitable area and take any suitable shape.

The first substrate 20 can include any suitable material is further described herein. Also, the substrate 20 can have any suitable dimensions. Further, the first substrate 20 can take any suitable shape in a plane parallel to the first major surface 14 of the backing layer 12, e.g., elliptical, rectilinear, etc. The first substrate 20 can have any suitable number of sides, e.g., one, two, three, four, five, six, seven, or more sides. In one or more embodiments, the first substrate 20 can take a polygonal shape. As used herein, the term “polygonal” refers to a planar shape having at least three straight sides and angles. In one or more embodiments, the polygonal shape of the first substrate 20 can have no greater than seven sides. In one or more embodiments, the polygonal shape of the first substrate 20 can include a diamond shape. The first substrate 20 can take the shape of a regular polygon or an irregular polygon. As used herein, the term “regular polygon” refers to a polygon that is equal angular (i.e., all angles are equal) and equilateral (i.e., all sides have the same length). Further, as used herein, the term “irregular polygon” is any polygon that is not a regular polygon.

Any suitable number of first microneedles 22 can be disposed on the substrate 20. Further, the first microneedles 22 can be disposed in any suitable arrangement on the first substrate 20. In one or more embodiments, the first microneedles 22 are disposed in an array that can be random, pseudorandom, or ordered. Each microneedle of the first microneedles 22 can take any suitable shape and have any suitable dimensions as is further described herein. In one or more embodiments, the first microneedles 22 can be arranged in uniformly spaced rows. When arranged in rows, the rows can be arranged so that the first microneedles 22 are aligned or offset. In one or more embodiments, the first microneedles 22 can be arranged in a polygonal pattern such as a triangle, square, rectangle, pentagon, hexagon, heptagon, octagon, or trapezoid. In one or more embodiments, the first microneedles 22 can be arranged in a circular or oval pattern.

The first microneedles 22 can be disposed on the substrate 20 using any suitable technique. In one or more embodiments, the first microneedles 22 can be molded with the substrate 20 such that microneedles and the substrate or a unitary component. In one or more embodiments, the first microneedles 22 can be formed separately (either completely or partially) from the first substrate 20 and disposed on the first substrate using a suitable technique, e.g., cast and cure.

Although not shown, a first medicament can be disposed on at least one first microneedle of the first microneedles 22 using any suitable technique. Any suitable medicament can be utilized, e.g., one or more of the medicaments described herein.

The plurality of discrete arrays 16 can further include the second discrete array 24. The second discrete array 24 includes the second substrate 26 and second microneedles 28 disposed on the second substrate. The second substrate 26 can include any suitable substrate described herein, e.g. first substrate 20 of first discrete array 18. Further, the second microneedles 28 can include any suitable microneedles described herein, e.g., first microneedles 22 of first discrete array 18. In one or more embodiments, a second medicament can be disposed on at least one second microneedle of the second microneedles 28 using any suitable technique. Any suitable medicament can be utilized, e.g., one or more of the medicaments described herein

The plurality of discrete arrays 16 can include any suitable number and type of discrete arrays. In one or more embodiments, the discrete arrays 16 can include only first discrete arrays 18. In one or more embodiments, the discrete arrays 16 can include one or more first discrete arrays 18 and one or more second discrete arrays 24. Further, in one or more embodiments, the discrete arrays 16 can include one or more first discrete arrays 18 and second discrete arrays 24, and one or more third discrete arrays 40. The third discrete array 40 can include a third substrate 42 and third microneedles 44 disposed on the third substrate. The device 10 can include any suitable number of types of discrete arrays, first, second, third, fourth, fifth or more discrete arrays.

As is further described herein, the first discrete array 18 can include a first characteristic and the second discrete array 24 can include a second characteristic different from the first characteristic. In embodiments that include third discrete arrays 40, the third discrete array can include a third characteristic that can be different from at least one of the first characteristic of the first discrete array 18 or the second characteristic of the second discrete array 24.

The discrete arrays 16 can be disposed on the backing layer 12 (or directly onto the skin without a backing layer) such that individual arrays are not connected to one or more adjacent arrays. In one or more embodiments, one or more of the discrete arrays 16 can be connected using any suitable technique. For example, as shown in FIG. 1, the first discrete array 18 includes one or more first alignment features 50 that extend from a perimeter 52 of the first discrete array. Similarly, the second discrete array 24 can include one or more second alignment features 58 that extend from a perimeter 60 of the second discrete array. The alignment features 50, 58 can include any suitable features that are configured to align and connect adjacent discrete arrays. For example, as shown in FIG. 3, the first alignment features 50 of the first discrete array 18 include one or more tabs 54 and one or more slots 56. The tabs 54 can be configured to connect with one or more slots (not shown) of the second alignment features 58 of the second discrete arrays 24. The tabs 54 can take any suitable shape and have any suitable dimensions. As shown in FIG. 3, each tab 54 has a thickness measured in a direction orthogonal to the upper surface 46 of the first substrate 20 that is less than a thickness of the first substrate. In one or more embodiments, at least one tab 54 can have a thickness that is equal to or greater than the thickness of the first substrate. Further, the slots 56 can take any suitable shape and have any suitable dimensions that that they are each configured to connect to a tab of an adjacent discrete array 16. As shown in FIG. 3, the slot 56 has a thickness that is less than the thickness of the first substrate 20. Each discrete array 16 can include any suitable number of alignment features having any suitable configuration. Further, the third discrete array 40 can include any suitable alignment features.

For example, FIG. 6 is a schematic perspective view of another embodiment of a drug delivery device 200. All design considerations and possibilities described herein regarding drug delivery device 10 of FIGS. 1-5 apply equally to drug delivery device 200 of FIG. 6. The device 200 includes a plurality of discrete arrays 216 that each take hexagonal shape. Extending from a perimeter 252 of one or more of the discrete arrays 216 are alignment features 250. Such alignment features include one or more tabs 254 and one or more slots 256, where each tab extends from a substrate 220 of the discrete array 216, and each slot is disposed in the substrate. Each tab 254 has a thickness that is less than a thickness of the substrate 220, and each slot 256 has a thickness that is less than the thickness of the substrate 220. The slots 256 are configured to connect to a tab 254 of an adjacent discrete array substrate 220 to provide cavities that are configured to receive the tabs, which have a depth that is no greater than a depth of the cavity.

Returning to FIGS. 1-5, the device 10 can include one or more openings 76 disposed through at least one of the discrete arrays 16. As shown in FIG. 2, the opening 76 is disposed through the second substrate 26 of the second array 24. In one or more embodiments, the device 10 can include one or more openings disposed through one or more of the discrete arrays 16. Each of the openings 76 can include any suitable cross-sectional shape and have any suitable dimensions.

The discrete arrays 16 can be disposed in any suitable relationship or pattern on the backing layer 12 (or directly on the skin without a backing layer). For example, the discrete arrays 16 can form a tessellated pattern on the backing layer 12. Further, the discrete arrays 16 can be disposed such that two or more arrays are in contact. For example, in one or more embodiments, the first discrete array 18 can be in contact with the second discrete array 24. Further, two or more of the discrete arrays 16 can disposed such that there is a fluid channel 78 disposed between the arrays. For example, in the embodiment illustrated in FIG. 2, the fluid channel 78 can be disposed between the first discrete array 18 and the second discrete array 24. The fluid channel 78 can take any suitable shape and have any suitable dimensions. Further, the device 10 can include any suitable number of fluid channels 78 disposed between adjacent discrete arrays 16.

In general, discrete arrays useful for practicing the present disclosure can have a variety of characteristics, such as those described in the following patents and patent applications. One embodiment for the microneedles of one or more of the discrete arrays 16 includes the structures disclosed in U.S. Patent Application Publication No. 2005/0261631 (Clarke et al.), which describes microneedles having a truncated tapered shape and a controlled aspect ratio. Another embodiment for the microneedles discrete arrays includes the structures disclosed in U.S. Pat. No. 6,091,975 (Daddona et al.), which describes blade-like microprotrusions for piercing the skin. Still another embodiment of microneedles includes the structures disclosed in U.S. Pat. No. 6,312,612 (Sherman et al.), which describes tapered structures having a hollow central channel. Yet still another embodiment of microneedles includes the structures disclosed in U.S. Pat. No. 6,379,324 (Gartstein et al.), which describes hollow microneedles having at least one longitudinal blade at the top surface of the tip of the microneedle. A further embodiment of microneedles includes the structures disclosed in U.S. Patent Application Publication Nos. US2012/0123387(Gonzalez et al.) and US2011/0213335 (Burton et al.), which both describe hollow microneedles. A still further embodiment of microneedles includes the structures disclosed in U.S. Pat. No. 6,558,361 (Yeshurun) and 7,648,484 (Yeshurun et al.), which both describe hollow microneedle arrays and methods of manufacturing thereof.

Various embodiments of microneedles that can be employed in the discrete arrays of the present disclosure are also described in PCT Publication No. WO2012/074576 (Duan et al.), which describes liquid crystalline polymer (LCP) microneedles; and PCT Publication No. WO2012/122162 (Zhang et al.), which describes a variety of different types and compositions of microneedles.

In one or more embodiments, the microneedle material can be (or include) silicon, glass, or a metal such as stainless steel, titanium, or nickel titanium alloy. In one or more embodiments, the microneedle material can be (or include) a polymeric material, e.g., a medical grade polymeric material. Exemplary types of medical grade polymeric materials include polycarbonate, liquid crystalline polymer (LCP), polyether ether ketone (PEEK), cyclic olefin copolymer (COC), polybutylene terephthalate (PBT).

In one or more embodiments, the microneedle material can be (or include) a biodegradable polymeric material, e.g., a medical grade biodegradable polymeric material. Exemplary types of medical grade biodegradable materials include polylactic acid (PLA), polyglycolic acid (PGA), PGA and PLA copolymer, polyester-amide polymer (PEA).

In one or more embodiments, the microneedles can be prepared from a dissolvable, degradable, or disintegradable material referred to herein as “dissolvable microneedles.” A dissolvable, degradable, or disintegradable material is any solid material that dissolves, degrades, or disintegrates during use. In particular, a “dissolvable microneedle” dissolves, degrades, or disintegrates sufficiently in the tissue underlying the stratum corneum to allow a therapeutic agent to be released into the tissue. The therapeutic agent may be coated on or incorporated into a dissolvable microneedle. In one or more embodiments, the dissolvable material is selected from a carbohydrate or a sugar. In one or more embodiments, the dissolvable material is polyvinyl pyrrolidone (PVP). In one or more embodiments, the dissolvable material is selected from the group consisting of hyaluronic acid, carboxymethylcellulose, hydroxypropylmethylcellulose, methylcellulose, polyvinyl alcohol, sucrose, glucose, dextran, trehalose, maltodextrin, and a combination thereof.

In one or more embodiments, the microneedles can be made from (or include) a combination of two or more of any of the above-mentioned materials. For example, the tip of a microneedle may be a dissolvable material, while the remainder of the microneedle is a medical grade polymeric material.

A microneedle or the plurality of microneedles of a discrete array can have a variety of shapes that are capable of piercing the stratum corneum. In one or more embodiments, one or more of the microneedles (e.g., first microneedles 22) can have a square pyramidal shape, triangular pyramidal shape, stepped pyramidal shape, conical shape, microblade shape, or the shape of a hypodermic needle. In one or more embodiments, one or more of the microneedles can have a square pyramidal shape. In one or more embodiments, one or more of the microneedles can have a triangular pyramidal shape. In one or more embodiments, one or more of the microneedles can have a stepped pyramidal shape. In one or more embodiments, one or more of the microneedles can have a conical shape. In one or more embodiments, one or more of the microneedles can have a microblade shape. In one or more embodiments, one or more of the microneedles can have the shape of a hypodermic needle. The shape can be symmetric or asymmetric. The shape can be truncated (for example, the microneedles can have a truncated pyramid shape or truncated cone shape).

In one or more embodiments, one or more of the microneedles of a discrete array are solid microneedles (that is, the microneedles are solid throughout). In one or more embodiments, the solid microneedles can have a square pyramidal shape, triangular pyramidal shape, stepped pyramidal shape, conical shape, or microblade shape.

In one or more embodiments, one or more of the microneedles are hollow microneedles (that is, the microneedles contain a hollow bore through the microneedle). The hollow bore can be from the base of the microneedle to the tip of the microneedle or the bore can be from the base of the microneedle to a position offset from the tip of the microneedle. In one or more embodiments, one or more of the hollow microneedles can have a conical shape, cylindrical shape, square pyramidal shape, triangular pyramidal shape, or the shape of a hypodermic needle.

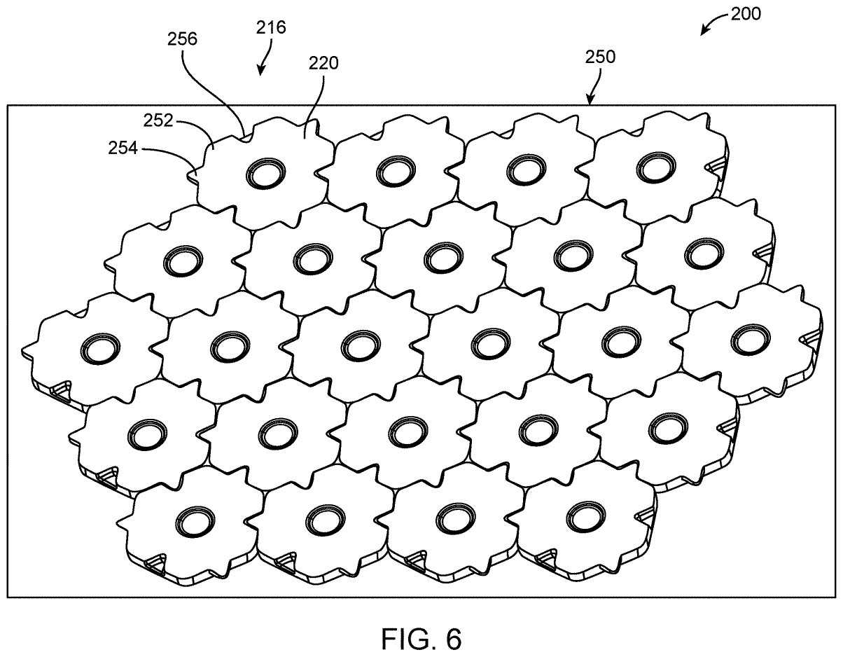

FIG. 5 is a schematic cross-section view of a portion of the first discrete array 18 that includes first microneedles 22 disposed on the first substrate 20. Each microneedle 22 has a height h, which is the length from the tip of the microneedle to the microneedle base at the first substrate 20. Either the height of a single microneedle 22 or the average height of all microneedles of the first discrete array 18 can be referred to as the height of the microneedle, h. In one or more embodiments, each of the first microneedles 22 (or the average of all of the first microneedles) has a height of about 100 to about 3000 micrometers. In one or more embodiments, each of the first microneedles 22 (or the average of all of the first microneedles) has a height of less than about 3000 micrometers. In one or more embodiments, each of the first microneedles 22 (or the average of all of the first microneedles) has a height of at least about 100 micrometers.

In one or more embodiments employing solid microneedles, each of the solid microneedles (or the average of all of the solid microneedles) has a height of about 100 to about 1500 micrometers. In one or more embodiments employing hollow microneedles, each of the hollow microneedles (or the average of all of the hollow microneedles) has a height of about 100 to about 3000 micrometers.

A single microneedle or the microneedles of the discrete arrays 16 can also be characterized by their aspect ratio. The aspect ratio of a microneedle is the ratio of the height of the microneedle, h to the width (at the base of the microneedle), w (as shown in FIG. 5). The aspect ratio can be presented as h:w. Each microneedle of a discrete array can have any suitable aspect ratio. In one or more embodiments, each of the microneedles of a discrete array (or the average of all the microneedles of the discrete array) has an aspect ratio in a range of 2:1 to 5:1.

Each of the discrete arrays 16 can include any suitable number of microneedles. In one or more embodiments, a discrete array includes about 100 to about 1500 microneedles per cm² of the array. In one or more embodiments employing solid microneedles, a discrete array of solid microneedles can include about 100 to about 1500 solid microneedles per cm² of the array. In one or more embodiments employing hollow microneedles, the discrete array of hollow microneedles contains about 3 to about 30 hollow microneedles per cm² of the array.

In one or more embodiments, each of the microneedles (or the average of all of the microneedles) of a discrete array can penetrate into the skin to a depth of about 50 to about 1500 micrometers. It will be appreciated that the depth of penetration (DOP) of each of the microneedles (or the average of all of the microneedles) of a discrete array may not be the full length of the microneedles themselves.

Further, any suitable area of the discrete array substrate can be covered with microneedles. In one or more embodiments, about 0.1 cm² to about 20 cm² of the surface area of the discrete array substrate is covered with microneedles.

In one or more embodiments, the microneedles of the present disclosure can be disposed over substantially the entire surface of the discrete array. In one or more embodiments, a portion of the substrate of the discrete array may not be provided with microneedles (that is, a portion of the substrate is non-structured). For example, as shown in FIG. 3, the first portion 80 of the substrate 20 of the first discrete array includes first microneedles 22 and the second portion 82 does not include first microneedles to provide a non-structured surface of the substrate. In one or more of these embodiments, the non-structured surface can have an area of more than about 1 percent and less than about 75 percent of the total area of the substrate surface that faces the skin surface. In another of these embodiments, the non-structured surface has an area of more than about 0.65 cm² (0.10 square inch) to less than about 6.5 cm² (1 square inch).

For hollow microneedles, a hollow channel or bore extends through the substrate and microneedles. In one or more embodiments, the bore exits at a channel opening at or near the tip of the hollow microneedle. In one or more embodiments, the channel can exit at an opening near the tip of the hollow microneedle. In one or more embodiments, the channel or bore continues along a central axis of the microneedle but exits similar to a hypodermic needle on a sloping side-wall of the microneedle to help prevent blockage of the channel by tissue upon insertion. In one or more embodiments, the diameter of the channel bore is about 10 to about 200 micrometers. In one or more embodiments, the diameter of the channel bore is about 10 to about 150 micrometers. In still other embodiments, the diameter of the channel bore is about 30 to about 60 micrometers. In one or more embodiments of hollow microneedles, the average cross-sectional area of the channel bore is about 75 to about 32,000 micrometers.

In one or more embodiments of hollow microneedle discrete arrays, the average spacing between adjacent microneedles (as measured from microneedle tip to microneedle tip) is between about 0.7 mm and about 20 mm. In one or more embodiments of hollow microneedle arrays, the average spacing between adjacent microneedles (as measured from microneedle tip to microneedle tip) is greater than about 0.7 mm. In one or more embodiments of hollow microneedle arrays, the average spacing between adjacent microneedles is less than about 20 mm.

In one or more embodiments of solid microneedle discrete arrays, the average spacing between adjacent microneedles (as measured from microneedle tip to microneedle tip) is between about 200 micrometers and about 2000 micrometers. In one or more embodiments of solid microneedle discrete arrays, the average spacing between adjacent microneedles is greater than about 200 micrometers. In one or more embodiments of solid microneedle discrete arrays, the average spacing between adjacent microneedles is less than about 2000 micrometers.

The discrete arrays can be manufactured in any suitable way such as by injection molding, compression molding, metal injection molding, stamping, photolithography, or extrusion. In one more embodiments, hollow microneedle discrete arrays can be made by injection molding of a polymer such as medical grade polycarbonate or LCP, followed by laser drilling to form the channels of the microneedles.

As mentioned herein, in one or more embodiments, active ingredients or agents (e.g., drugs) can be delivered via the microneedles of the discrete arrays (e.g., via solid or hollow microneedles, as described herein). Examples of pharmaceutically active agents (also referred to as “drugs”) that can be incorporated into the discrete arrays of the present disclosure are those capable of local or systemic effect when administered to the skin. Some examples include buprenorphine, clonidine, diclofenac, estradiol, granisetron, isosorbide dinitrate, levonorgestrel, lidocaine, methylphenidate, nicotine, nitroglycerine, oxybutynin, rivastigmine, rotigotine, scopolamine, selegiline, testosterone, tulobuterol, and fentanyl, which are commercially available in the form of transdermal devices. Other examples include antiinflammatory drugs, both steroidal (e.g., hydrocortisone, prednisolone, triamcinolone) and nonsteroidal (e.g., naproxen, piroxicam); bacteriostatic agents (e.g., chlorhexidine, hexylresorcinol); antibacterials (e.g., penicillins such as penicillin V, cephalosporins such as cephalexin, erythromycin, tetracycline, gentamycin, sulfathiazole, nitrofurantoin, and quinolones such as norfloxacin, flumequine, and ibafloxacin); antiprotazoals (e.g., metronidazole); antifungals (e.g., nystatin), coronary vasodilators; calcium channel blockers (e.g., nifedipine, diltiazem); bronchodilators (e.g., theophylline, pirbuterol, salmeterol, isoproterenol); enzyme inhibitors such as collagenase inhibitors, protease inhibitors, acetylcholinesterase inhibitors (e.g., donepezil), elastase inhibitors, lipoxygenase inhibitors (e.g., A64077), and angiotensin converting enzyme inhibitors (e.g., captopril, lisinopril); other antihypertensives (e.g., propranolol); leukotriene antagonists (e.g., ICI204,219); anti-ulceratives such as H2 antagonists; steroidal hormones (e.g., progesterone); antivirals and/or immunomodulators (e.g., 1-isobutyl-1H-imidazo[4,5-c]quinolin -4-amine, 1-(2-hydroxy-2-methylpropyl)-1H-imidazo[4,5-c]quinolin-4-amine, N-[4-(4-amino-2-ethyl-1H-imidazo[4,5-c]quinolin-1-yl)butyl]methanesulfonamide, and acyclovir); local anesthetics (e.g., benzocaine, propofol, tetracaine, prilocaine); cardiotonics (e.g., digitalis, digoxin); antitussives (e.g., codeine, dextromethorphan); antihistamines (e.g., diphenhydramine, chlorpheniramine, terfenadine); narcotic analgesics (e.g., morphine, fentanyl citrate, sufentanil, hydromorphone hydrochloride); peptide hormones (e.g., human or animal growth hormones, LHRH, parathyroid hormones); cardioactive products such as atriopeptides; antidiabetic agents (e.g., insulin, exanatide); enzymes (e.g., anti-plaque enzymes, lysozyme, dextranase); antinauseants; anticonvulsants (e.g., carbamazine); immunosuppressives (e.g., cyclosporine); psychotherapeutics (e.g., diazepam); sedatives (e.g., phenobarbital), anticoagulants (e.g., heparin, enoxaparina sodium); analgesics (e.g., acetaminophen); antimigraine agents (e.g., ergotamine, melatonin, sumatriptan, zolmitriptan); antiarrhythmic agents (e.g., flecainide); antiemetics (e.g., metaclopromide, ondansetron, granisetron hydrochloride); anticancer agents (e.g., methotrexate); neurologic agents such as anxiolytic drugs; hemostatics; anti-obesity agents; dopamine agonists (e.g., apomorphine); GnRH agonists (e.g., leuprolide, goserelin, nafarelin); fertility hormones (e.g., hCG, hMG, urofollitropin); interferons (e.g., interferon-alpha, interferon-beta, interferon-gamma, pegylated interferon-alpha); and the like, as well as pharmaceutically acceptable salts and esters thereof. The amount of drug that constitutes a therapeutically effective amount can be readily determined by those skilled in the art with due consideration of the particular drug, the particular carrier, and the desired therapeutic effect.

In one or more embodiments, peptide therapeutic agents (natural, synthetic, or recombinant) can be delivered via the microneedles of the discrete arrays (e.g., via solid or hollow microneedles, as described below). Examples of peptide therapeutic agents that can be incorporated into the discrete arrays of the present disclosure include parathyroid hormone (PTH), parathyroid hormone related protein (PTHrP), calcitonin, lysozyme, insulin, insulinotropic analogs, glatiramer acetate, goserelin acetate, somatostatin, octreotide, leuprolide, vasopressin, desmopressin, thymosin alpha-1, atrial natriuretic peptide (ANP), endorphin, vascular endothelial growth factor (VEGF), fibroblast-growth factor (FGF), erythropoietin (EPO), bone morphogenetic proteins (BMPs), epidermal growth factor (EFG), granulocyte colony-stimulating factor (G-CSF), granulocyte macrophage colony stimulating factor (GM-CSF), insulin-like growth factor (IGF), platelet-derived growth factor (PDGF), growth hormone release hormone (GHRH), dornase alfa, tissue plasminogen activator (tPA), urokinase, ANP clearance inhibitors, lutenizing hormone releasing hormone (LHRH), melanocyte stimulating hormones (alpha & beta MSH), pituitary hormones (hGH), adrenocorticotropic hormone (ACTH), human chorionic gonadotropin (hCG), streptokinase, interleukins (e.g. IL-2, IL-4, IL-10, IL-12, IL-15, IL-18), protein C, protein S, angiotensin, angiogenin, endothelins, pentigetide, brain natriuretic peptide (BNP), neuropeptide Y, islet amyloid polypeptide (IAPP), vasoactive intestinal peptide (VIP), hirudin, glucagon, oxytocin, and derivatives of any of the foregoing peptide therapeutic agents.

In one or more embodiments, drugs that are of a large molecular weight may be delivered transdermally. Increasing molecular weight of a drug typically can cause a decrease in unassisted transdermal delivery. Examples of such large molecules include proteins, peptides, nucleotide sequences, monoclonal antibodies, vaccines, polysaccharides, such as heparin, and antibiotics, such as ceftriaxone. Examples of suitable vaccines include therapeutic cancer vaccine, anthrax vaccine, flu vaccine, Lyme disease vaccine, rabies vaccine, measles vaccine, mumps vaccine, chicken pox vaccine, small pox vaccine, hepatitis vaccine, hepatitis A vaccine, hepatitis B vaccine, hepatitis C vaccine, pertussis vaccine, rubella vaccine, diphtheria vaccine, encephalitis vaccine, Japanese encephalitis vaccine, respiratory syncytial virus vaccine, yellow fever vaccine, recombinant protein vaccine, DNA vaccines, polio vaccine, herpes vaccine, human papilloma virus vaccine, pneumococcal vaccine, meningitis vaccine, whooping cough vaccine, tetanus vaccine, typhoid fever vaccine, cholera vaccine, tuberculosis vaccine, severe acute respiratory syndrome (SARS) vaccine, HSV-1 vaccine, HSV-2 vaccine, HIV vaccine and combinations thereof. The term “vaccine” thus includes, without limitation, antigens in the forms of proteins, polysaccharides, oligosaccharides, or weakened or killed viruses. Additional examples of suitable vaccines and vaccine adjuvants are described in U.S. Patent Application Publication No. 2004/0049150 (Dalton et al.).

In one or more embodiments, small-molecule drugs that are otherwise difficult or impossible to deliver by passive transdermal delivery may be used. Examples of such molecules include salt forms; ionic molecules, such as bisphosphonates, including sodium alendronate or pamedronate; and molecules with physicochemical properties that are not conducive to passive transdermal delivery.

As mentioned herein, each discrete array of the plurality of discrete arrays 16 can include one or more characteristics that are the same as or different from one or more characteristics of one or more additional arrays of the device 10. For example, the first discrete array 18 of the device 10 can include a first characteristic and the second discrete array 24 can include a second characteristic. In one or more embodiments, the first characteristic can be the same as the second characteristic. In one or more embodiments, the first characteristic can be different from the second characteristic. Further, in embodiments where the device 10 includes one or more third discrete arrays 40, such third discrete arrays can include a third characteristic that is the same as or different from at least one of the first characteristic or second characteristic.

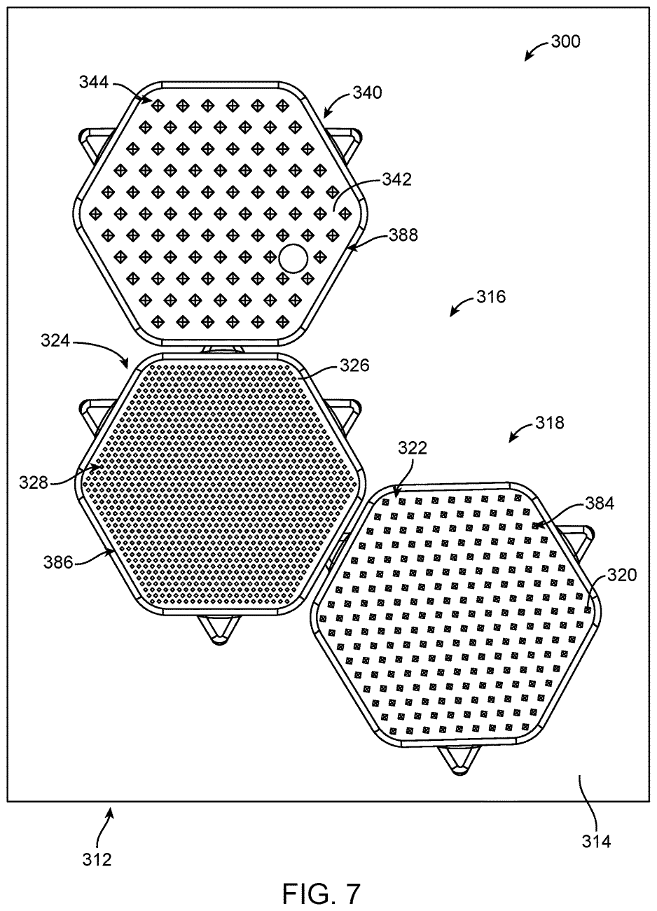

For example, FIG. 7 is a schematic plan view of a portion of another embodiment of a drug delivery device 300. All design considerations and possibilities described herein regarding the drug delivery device 10 of FIGS. 1-5 apply equally to the drug delivery device 300 of FIG. 7. The device 300 includes a backing layer 312 and a plurality of discrete arrays 316 disposed on a first major surface 314 of the backing layer. The plurality of discrete arrays 316 includes a first discrete array 318 and a second discrete array 324. The first discrete array 318 includes a first substrate 320 and first microneedles 322 disposed on the first substrate. Further, the second discrete array 324 includes a second substrate 326 and second microneedles 328 disposed on the second substrate. The plurality of discrete arrays 316 also includes a third discrete array 340 that includes a third substrate 342 and third microneedles 344 disposed on the third substrate. Although only one of each of the first, second, and third discrete arrays 318, 324, and 340 are shown, the device 300 can include any suitable number of each of the first, second, third discrete arrays disposed in any suitable arrangement on the first major surface 314 of the backing layer 312. In one or more embodiments, the device 300 can include one or more of fourth, fifth, sixth, or more discrete arrays.

The first discrete array 318 includes a first characteristic, the second discrete array 324 includes a second characteristic, and the third discrete array 340 includes a third characteristic. The first, second, and third characteristics can include any suitable properties of the discrete arrays 316. The characteristics of each of the discrete arrays 316 can be any suitable property, e.g., an arrangement of microneedles, shape of at least one microneedle, cross-sectional shape of at least one microneedle, aspect ratio of at least one microneedle, medicament disposed on at least one microneedle, shape of the array substrate, number of microneedles, type of microneedle (e.g., solid, hollow, dissolvable), etc.

For example, the first characteristic of the first discrete array 318 can include a first arrangement 384 of the first microneedles 322, the second characteristic can include a second arrangement 386 of the second microneedles 328, and the third characteristic of the third discrete array 340 can include a third arrangement 388 of third microneedles. The first arrangement 384 can be the same as or different from the second arrangement 386 and the third arrangement 388.

Further, for example, the first characteristic of the first discrete array 318 can include a shape of at least one first microneedle 322, the second characteristic of the second discrete array 324 can include a shape of at least one second microneedle 328, and the third characteristic of the third discrete array 340 can include a shape of at least one third microneedle 344. In one or more embodiments, the first characteristic can include a first cross-sectional shape in a plane parallel to the backing layer 312 of at least one first microneedle 322, the second characteristic can include a second cross-sectional shape in the plane parallel to the backing layer of at least one second microneedle 328, and the third characteristic can include a third cross-sectional shape in the plane parallel to the backing layer of at least one third microneedle 344.

Further, for example, the first characteristic can include an aspect ratio of at least one first microneedle 322, the second characteristic can include an aspect ratio of at least one second microneedle 328, and the third characteristic can include an aspect ratio of at least one third microneedle 344.

In one or more embodiments, the first characteristic can include a first medicament disposed on at least one first microneedle 322, the second characteristic can include a second medicament disposed on at least one second microneedle 328, and the third characteristic can include a third medicament disposed on at least one third microneedle 344. In one or more embodiments, the first characteristic can include a first quantity of a medicament disposed on at least one first microneedle 322, the second characteristic can include a second quantity of the medicament disposed on at least one second microneedle 328, and the third characteristic can include a third quantity of the medicament disposed on at least one third microneedle 344.

In one or more embodiments, the first characteristic can include a first shape of the first substrate 320 in a plane parallel to the backing layer 312, the second characteristic can include a second shape of the second substrate 326 in the plane parallel to the backing layer, and the third characteristic can include a shape of the third substrate 342 in the plane parallel to the backing layer. In one or more embodiments, the first characteristic can include a first shape of the first substrate 320 in a plane orthogonal to the backing layer 312, the second characteristic can include a second shape of the second substrate 326 in the plane orthogonal to the backing layer, and the third characteristic can include a shape of the third substrate 342 in the plane orthogonal to the backing layer.

Any suitable technique can be utilized to dispose the device 10 onto the skin of the user. For example, a microarray applicator can be configured to deliver the device 10. Any suitable microarray applicator can be utilized, e.g., one or more embodiments of applicators described in U.S. Provisional Application No. 63/422,965, filed Nov. 5, 2022, and entitled MICRONEEDLE ARRAY APPLICATOR AND SYSTEM; U.S. patent application Ser. No. 16/956,222, filed Jun. 19, 2020, and entitled MICROARRAY APPLICATION; and U.S. Pat. No. 11,458,289, issued Oct. 4, 2022, and entitled APPLICATOR FOR APPLYING A MICRONEEDLE ARRAY TO SKIN.

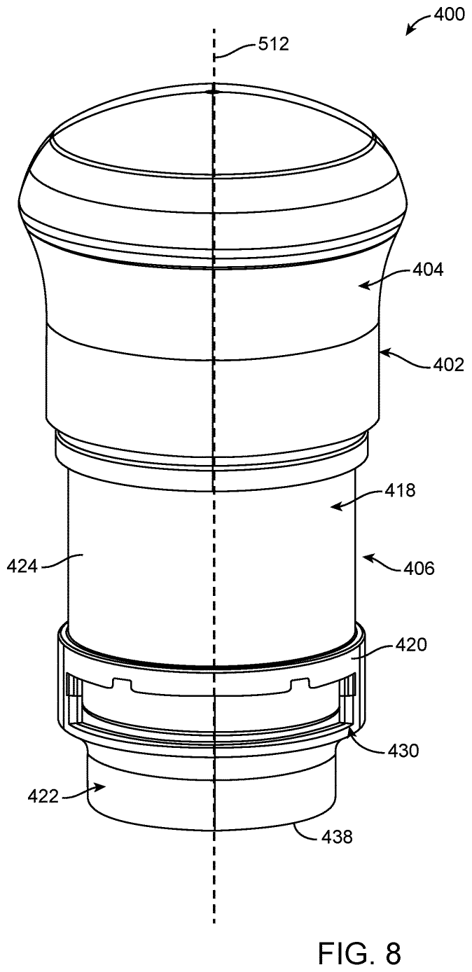

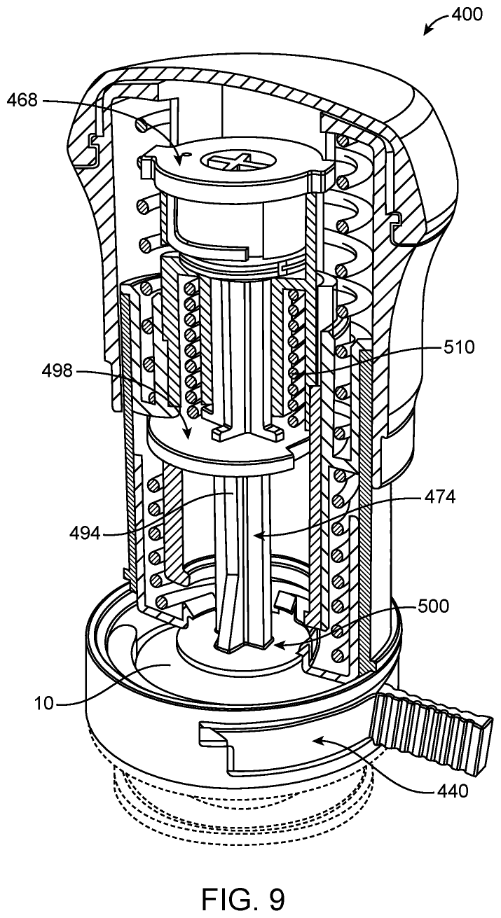

One exemplary applicator 400 is illustrated in FIGS. 8-9 and further described U.S. U.S. Pat. No. 11,458,289. The applicator 400 includes a body 402 having an upper housing 404 and a lower housing 406. The lower housing 406 includes an upper portion 418, a middle portion 420, and a lower portion 422. The upper portion 418 of the lower housing 406 includes an outer surface 424 that can have a smooth surface finish or other finish with low surface energy to enable sliding engagement with an inner surface of upper housing 404. Middle portion 420 of the lower housing 406 can include a slot 430 or opening for insertion of a microneedle array carrier 440 (FIG. 9). Lower portion 422 includes a lower surface 438 on its underside.

The applicator 400 includes a plunger 474 that can include a plunger post 494, a first plunger disc 498, and a second plunger disc 500 proximate a lower end thereof. The applicator 400 further includes a firing spring 510 disposed proximate the first plunger disc 498 presented on plunger 474. In one or more embodiments, biasing members other than compression springs, such as leaf springs, rubber members, or structures, can be used in place of or alternative to compression springs.

In use, the lower surface 438 of lower portion 422 of lower housing 406 is placed in contact with the skin or another surface, such as a sterile or sanitary surface “S.” The upper housing 404 is pushed down axially along an axis 512 of applicator 400 such that the inner surface of the upper housing 404 slides over the outer surface 424 of lower housing 406 and firing spring 510 is compressed and, thus, energized. This energized firing spring 510 is later used to drive the drug delivery device 10 from the applicator 400.

As shown in FIG. 9, the drug delivery device 10 can be inserted into the slot 430 of the applicator 400 via a microneedle array carrier 440. Such carrier 440 is further described, e.g., in U.S. Pat. No. 11,458,289. Once microneedle array carrier 440, with drug delivery device 10, has been inserted into slot 430, the drug delivery device can be applied to the skin by axially compressing, i.e., pushing down, on upper housing 404 again. That causes latch 468 to rotate in a clockwise fashion, which releases the spring-loaded plunger 474, and the firing spring 510 releases to its uncompressed state. The second plunger disc 500 of spring-loaded plunger 474 can be positioned to rest directly against or very close to the second major surface 15 of the drug delivery device 10 once primed or come into direct contact of come very close to bottom surface second major surface of the drug delivery device when the upper housing 404 is again pressed axially down, but before the plunger 474 is released (i.e., the applicator 400 is fired). By having this configuration, a user can inhibit instability issues that might arise if a plunger were to accelerate and hit a microneedle array assembly to release it from the carrier while still in the applicator. Instead, the plunger 474 pushes the drug delivery device 10 from microneedle array carrier 440 as it begins to accelerate and reaches the desired impact speed about a time, or substantially simultaneously with, the drug delivery device contacting the skin.

In one or more embodiments, the applicator 400 can be configured to apply different impact and holding forces behind different discrete arrays to optimize depth of penetration. For example, the applicator 400 can be configured to apply a first impact force to a discrete array having a length h₁ of each microneedle and a second impact force to a discrete array having a length h₂ of each microneedle, where h₁ is greater than h₂. In one or more embodiments, the applicator 400 can be configured such that a lower surface of the second plunger disc 500 that contacts the drug delivery device 10 can have one or more non-parallel or non-uniform shapes that can provide variations in penetration depth to the microneedles of the various discrete arrays 16.

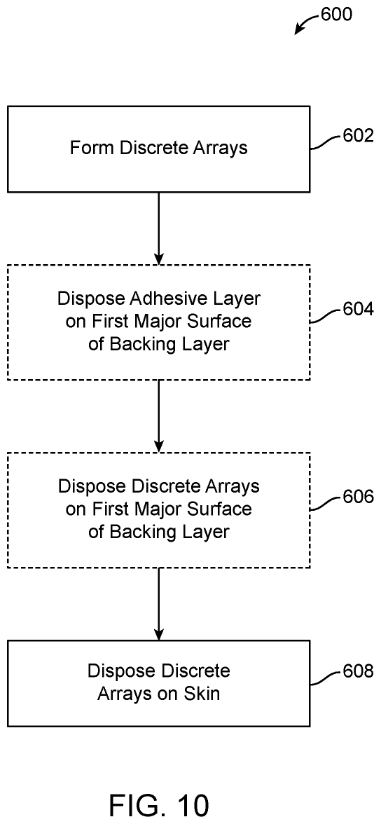

Various techniques can be utilized to form the drug delivery device 10. For example, FIG. 10 is a flowchart of one embodiment of a method 600 of forming the drug delivery device 10. Although described regarding device 10 of FIGS. 1-5, the method 600 can be utilized to form any suitable drug delivery device. At 602, the plurality of discrete arrays 16 can be formed using any suitable technique. The plurality of discrete arrays 16 includes the first discrete array 18 having the first substrate 20 and the first microneedles 22 disposed on the first substrate, and the second discrete array 24 having the second substrate 26 and the second microneedles 28 disposed on the second substrate 26. The first discrete array 18 includes the first characteristic, and the second discrete array 24 includes the second characteristic that is different from the first characteristic. The plurality of discrete arrays 16 can include any suitable number and type of discrete arrays. In one or more embodiments, the plurality of discrete arrays 16 can include the third discrete array 40 that includes the third substrate 42 and third microneedles 44 disposed on the third substrate, where the third discrete array includes a third characteristic different from the first characteristic and second characteristic. In one or more embodiments, at least one of the first discrete array 18 or the second discrete array 24 can be formed by microreplication.

The adhesive layer 30 can optionally be disposed on the first major surface 14 of the backing layer 12 at 604 using any suitable technique for embodiments where the plurality of discrete arrays 16 are disposed on the backing layer prior to be disposed on the skin of the user. At 606, the plurality of discrete arrays 16 can optionally be disposed on the first major surface 14 of the backing layer 12 when such backing layer is utilized. Any suitable technique can be utilized to dispose the discrete arrays 16 on the backing layer. In one or more embodiments, one or more of the discrete arrays 16 can be connected together prior to being disposed on the backing layer 12. At 608, the discrete arrays 16 can be disposed on the skin of the user using any suitable technique. In one or more embodiments, an applicator (e.g., applicator 400) can be utilized to dispose the discrete arrays 16 on the skin.

One or more openings can be disposed through at least one discrete array of the plurality of discrete arrays 16 using any suitable technique prior to disposing such arrays on the skin of the user. Further, one or more fluid channels 78 can be disposed between adjacent arrays of the discrete arrays 16 using any suitable technique. Any suitable number of channels 78 can be disposed between two or more discrete arrays of the plurality of discrete arrays 16.

The invention is defined in the claims; however, below there is provided a non-exhaustive listing of non-limiting examples. Any one or more of the features of these examples may be combined with any one or more features of another example, embodiment, or aspect described herein.

Example Ex1. A drug delivery device that includes a backing layer having a first major surface and a second major surface, and a plurality of discrete arrays disposed on the first major surface of the backing layer. The plurality of discrete arrays includes a first discrete array that has a first substrate and first microneedles disposed on the first substrate, and a second discrete array that has a second substrate and second microneedles disposed on the second substrate.

Example Ex2. The device of Ex1, where the first discrete array includes a first characteristic and the second discrete array includes a second characteristic different from the first characteristic.

Example Ex3. The device of Ex2, where the first characteristic includes a first arrangement of the first microneedles and the second characteristic includes a second arrangement of the second microneedles, where the first arrangement is different from the second arrangement,

Example Ex4. The device of any of Ex2 to Ex3, where the first characteristic includes a shape of at least one first microneedle and the second characteristic includes a shape of at least one second microneedle.

Example Ex5. The device of Ex4, where the at least one first microneedle includes a first cross-sectional shape in a plane parallel to the backing layer and the at least one second microneedle includes a second cross-sectional shape in the plane parallel to the backing layer.

Example Ex6. The device of any one of Ex2 to Ex5, where the first characteristic includes an aspect ratio of at least one first microneedle and the second characteristic includes an aspect ratio of at least one second microneedle.

Example Ex7. The device of any one of Ex2 to Ex6, where the first characteristic includes a first medicament disposed on at least one first microneedle and the second characteristic includes a second medicament disposed on at least one second microneedle.

Example Ex8. The device of any one of Ex2 to Ex7, where the first characteristic includes a first shape of the first substrate of the first discrete array in a plane parallel to the backing layer and the second characteristic includes a second shape of the second substrate of the second discrete array in the plane parallel to the backing layer.

Example Ex9. The device of Ex8, where at least one of the first shape of the first substrate or the second shape of the second substrate includes a polygonal shape.

Example Ex10. The device of Ex9, where the polygonal shape includes at least three sides.

Example Ex11. The device of any one of Ex9 to Ex10, where the polygonal shape includes no greater than seven sides.

Example Ex12. The device of any one of Ex9 to Ex11, where the polygonal shape includes a diamond shape.

Example Ex13. The device of any one of Ex2 to Ex12, further including a third discrete array disposed on the first major surface of the backing layer and including a third substrate and third microneedles disposed on the third substrate.

Example Ex14. The device of Ex13, where a third characteristic of the third discrete array is different from the first characteristic and the second characteristic.

Example Ex15. The device of any one of Ex1 to Ex14, where the plurality of discrete arrays form a tessellated pattern on the backing layer.

Example Ex16. The device of any one of Ex1 to Ex15, where the first discrete array is in contact with the second discrete array.

Example Ex17. The device of any one of Ex1 to Ex15, further including a fluid channel disposed between the first and second discrete arrays.

Example Ex18. The device of any one of Ex1 to Ex17, where at least one microneedle of the first or second microneedles includes a dissolvable material.

Example Ex19. The device of any one of Ex1 to Ex18, further including an opening disposed through at least one of the first substrate of the first discrete array or the second substrate of the second array.

Example Ex20. The device of any one of Ex1 to Ex19, where a first portion of the first substrate of the first discrete array includes an arrangement of first microneedles and a second portion of the first substrate does not include first microneedles.

Example Ex21. The device of any one of Ex1 to Ex20, where the first discrete array further includes one or more alignment features that are configured to be connected to one or more alignment features of the second discrete array such that the first discrete array is connected to the second discrete array.

Example Ex22. The device of any one of Ex1 to Ex21, further including an adhesive layer disposed on the first major surface of the backing layer such that the adhesive layer is between the first and plurality of discrete arrays and the first major surface of the backing layer.

Example Ex23. The device of Ex22, where a portion of the adhesive layer adjacent to a periphery of the backing layer is exposed such that the adhesive layer is configured to be adhered to skin of a user.

Example Ex24. The device of any one of Ex1 to Ex23, where the backing layer includes a polygonal shape in a plane parallel to the first major surface of the backing layer.

Example Ex25. The device of any one of Ex1 to Ex24, further including a release liner disposed over the plurality of discrete arrays such that the discrete arrays are disposed between the release liner and the backing layer.

Example Ex26. A method of forming a drug delivery device. The method includes forming a plurality of discrete arrays, where the plurality of discrete arrays includes a first discrete array having a first substrate and first microneedles disposed on the first substrate, and a second discrete array having a second substrate and second microneedles disposed on the second substrate. The first discrete array includes a first characteristic and the second discrete array includes a second characteristic different from the first characteristic. The method further includes disposing the plurality of discrete arrays on skin of a user.

Example Ex27. The method of Ex26, further including disposing the plurality of discrete arrays on a first major surface of a backing layer prior to disposing the plurality of discrete arrays on the skin of the user, where the backing layer further includes a second major surface.

Example Ex28. The method of Ex27, further including disposing an adhesive layer on the first major surface of the backing layer such that the adhesive layer is between the plurality of discrete arrays and the first major surface of the backing layer.

Example Ex29. The method of any one of Ex26 to Ex28, where forming the plurality of discrete arrays includes microreplicating at least one of the first discrete array or second discrete array.

Example Ex30. The method of any one of Ex26 to Ex29, further including disposing a first medicament on the first discrete array and a second medicament on the second discrete array, where the first characteristic includes the first medicament and the second characteristic includes the second medicament.

Example Ex31. The method of any one of Ex26 to Ex30, where forming the plurality of discrete arrays includes forming the first discrete array by disposing the first microneedles in a first arrangement on the first substrate and forming the second discrete array by disposing the second microneedles in a second arrangement on the second discrete array. The first characteristic includes the first arrangement and the second characteristic includes the second arrangement.

Example Ex32. The method of any of Ex26 to Ex31, further including disposing a fluid channel between the first discrete array and the second discrete array.

Example Ex33. The method of any one of Ex26 to Ex32, further including disposing an opening through at least one of the first substrate of the first discrete array or the second substrate of the second array.

Example Ex34. The method of any one of Ex28 to Ex33, where forming the plurality of discrete arrays further includes forming a third discrete array having a third substrate and third microneedles disposed on the third substrate, where the third discrete array includes a third characteristic different from the first characteristic and second characteristic.

Example Ex35. A drug delivery system including the drug delivery device of any one of Ex1 to Ex25 and an applicator configured to apply the drug delivery device to skin of a patient.

All references and publications cited herein are expressly incorporated herein by reference in their entirety into this disclosure, except to the extent they may directly contradict this disclosure. Illustrative embodiments of this disclosure are discussed and reference has been made to possible variations within the scope of this disclosure. These and other variations and modifications in the disclosure will be apparent to those skilled in the art without departing from the scope of the disclosure, and it should be understood that this disclosure is not limited to the illustrative embodiments set forth herein. Accordingly, the disclosure is to be limited only by the claims provided below.

1. A drug delivery device comprising:
a backing layer comprising a first major surface and a second major surface; and
a plurality of discrete arrays disposed on the first major surface of the backing layer, wherein the plurality of discrete arrays comprises a first discrete array comprising a first substrate and first microneedles disposed on the first substrate, and a second discrete array comprising a second substrate and second microneedles disposed on the second substrate.

2. The device of claim 1, wherein the first discrete array comprises a first characteristic and the second discrete array comprises a second characteristic different from the first characteristic.

3. The device of claim 2, wherein the first characteristic comprises a first arrangement of the first microneedles and the second characteristic comprises a second arrangement of the second microneedles, wherein the first arrangement is different from the second arrangement.

4. The device of claim 2, wherein the first characteristic comprises a shape of at least one first microneedle and the second characteristic comprises a shape of at least one second microneedle.

5. The device of claim 4, wherein the at least one first microneedle comprises a first cross-sectional shape in a plane parallel to the backing layer and the at least one second microneedle comprises a second cross-sectional shape in the plane parallel to the backing layer.

6. The device of claim 2, wherein the first characteristic comprises an aspect ratio of at least one first microneedle and the second characteristic comprises an aspect ratio of at least one second microneedle.

7. The device of claim 2, wherein the first characteristic comprises a first medicament disposed on at least one first microneedle and the second characteristic comprises a second medicament disposed on at least one second microneedle.

8. The device of claim 2, further comprising a third discrete array disposed on the first major surface of the backing layer and comprising a third substrate and third microneedles disposed on the third substrate.

9. The device of claim 1, wherein the plurality of discrete arrays form a tessellated pattern on the backing layer.

10. The device of claim 1, further comprising a fluid channel disposed between the first and second discrete arrays.

11. The device of claim 1, wherein the first discrete array further comprises one or more alignment features that are configured to be connected to one or more alignment features of the second discrete array such that the first discrete array is connected to the second discrete array.

12. The device of claim 1, further comprising an adhesive layer disposed on the first major surface of the backing layer such that the adhesive layer is between the plurality of discrete arrays and the first major surface of the backing layer.

13. The device of claim 12, wherein a portion of the adhesive layer adjacent to a periphery of the backing layer is exposed such that the adhesive layer is configured to be adhered to skin of a user.

14. The device of claim 1, further comprising a release liner disposed over the plurality of discrete arrays such that the discrete arrays are disposed between the release liner and the backing layer.

15. A method of forming a drug delivery device, the method comprising:
forming a plurality of discrete arrays, wherein the plurality of discrete arrays comprises a first discrete array comprising a first substrate and first microneedles disposed on the first substrate, and a second discrete array comprising a second substrate and second microneedles disposed on the second substrate, wherein the first discrete array comprises a first characteristic and the second discrete array comprises a second characteristic different from the first characteristic; and
disposing the plurality of discrete arrays on skin of a user.

16. The method of claim 15, further comprising disposing the plurality of discrete arrays on a first major surface of a backing layer prior to disposing the plurality of discrete arrays on the skin of the user, wherein the backing layer further comprises a second major surface.

17. The method of claim 16, further comprising disposing an adhesive layer on the first major surface of the backing layer such that the adhesive layer is between the plurality of discrete arrays and the first major surface of the backing layer.

18. The method of claim 15, further comprising disposing a first medicament on the first discrete array and a second medicament on the second discrete array, wherein the first characteristic comprises the first medicament and the second characteristic comprises the second medicament.

19. The method of claim 15, wherein forming the plurality of discrete arrays comprises forming the first discrete array by disposing the first microneedles in a first arrangement on the first substrate, and forming the second discrete array by disposing the second microneedles in a second arrangement on the second discrete array, wherein the first characteristic comprises the first arrangement and the second characteristic comprises the second arrangement.

20. A drug delivery system comprising the drug delivery device of claim 1 and an applicator configured to apply the drug delivery device to skin of a patient.
